SEQUENCE LISTING (4 sequences)

SEQ ID NO: 1 - DNA, 720 bp, Artificial Sequence
  Single chain antibody coding sequence Mel14
atggaagtgaagctggtggagtctgggggaggcttagtgaagcctggagggtccctgaaactctcctgtgcagcctctggattcactttcagtagctatgccatgtcttgggttcgccagactccagagaagagcctggagtgggtcgcatccattagtagtggtgatagcacctactatccagacagtgtgaagggccgattcaccatctccagagataatgccaggaacatcctgtacctgcaaatgagcagtctgaggtctgaggacacggccatgtattactgtgcaagaggcggatggttacactactttgactacgggggccaaggcaccactctcacagtctcctcaggcggagggggatccggtggtggcggatctggaggtggcggcagcgacatccagatgacacagtctccatcctcactgtctgcatctctgggaggcaaggtcaccatcacttgcaaggcaagccaagacattaacaagtatatagcttggtatcaacacaaacctggaaaaggtcctaggctgctcatgcattacacatctacattacagccaggcatcccatcaaggttcagtggaagtgggtctgggagagattattccttcagcatcagcaacctggagcctgaagatattgcaacttattattgtctacagtatgataatcttctcacgttcggaggggggaccaagctggaaataaaacgt

SEQ ID NO: 2 - protein, 240 aa, Artificial Sequence
  Single chain antibody sequence MEL14
MEVKLVESGGGLVKPGGSLKLSCAASGFTFSSYAMSWVRQTPEKSLEWVASISSGDSTYYPDSVKGRFTISRDNARNILYLQMSSLRSEDTAMYYCARGGWLHYFDYGGQGTTLTVSSGGGGSGGGGSGGGGSDIQMTQSPSSLSASLGGKVTITCKASQDINKYIAWYQHKPGKGPRLLMHYTSTLQPGIPSRFSGSGSGRDYSFSISNLEPEDIATYYCLQYDNLLTFGGGTKLEIKR

SEQ ID NO: 3 - DNA, 720 bp, Artificial Sequence
  disulphide stabilized (dsFv) Single chain antibody coding sequence Mel14 scFv
atggaagtgaagctggtggagtctgggggaggcttagtgaagcctggagggtccctgaaactctcctgtgcagcctctggattcactttcagtagctatgccatgtcttgggttcgccagactccagagaagtgcctggagtgggtcgcatccattagtagtggtgatagcacctactatccagacagtgtgaagggccgattcaccatctccagagataatgccaggaacatcctgtacctgcaaatgagcagtctgaggtctgaggacacggccatgtattactgtgcaagaggcggatggttacactactttgactacgggggccaaggcaccactctcacagtctcctcaggcggagggggatccggtggtggcggatctggaggtggcggcagcgacatccagatgacacagtctccatcctcactgtctgcatctctgggaggcaaggtcaccatcacttgcaaggcaagccaagacattaacaagtatatagcttggtatcaacacaaacctggaaaaggtcctaggctgctcatgcattacacatctacattacagccaggcatcccatcaaggttcagtggaagtgggtctgggagagattattccttcagcatcagcaacctggagcctgaagatattgcaacttattattgtctacagtatgataatcttctcacgttcggatgcgggaccaagctggaaataaaacgt

SEQ ID NO: 4 - protein, 240 aa, Artificial Sequence
  disulphide stabilized Single chain antibody sequence Mel14 scFv
MEVKLVESGGGLVKPGGSLKLSCAASGFTFSSYAMSWVRQTPEKCLEWVASISSGDSTYYPDSVKGRFTISRDNARNILYLQMSSLRSEDTAMYYCARGGWLHYFDYGGQGTTLTVSSGGGGSGGGGSGGGGSDIQMTQSPSSLSASLGGKVTITCKASQDINKYIAWYQHKPGKGPRLLMHYTSTLQPGIPSRFSGSGSGRDYSFSISNLEPEDIATYYCLQYDNLLTFGCGTKLEIKR

(19) United States
(12) Patent Application Publication
(10) Pub. No.: US 2010/0047164 A1
(43) Pub. Date: Feb. 25, 2010

(54) Anti-Tumor Antibodies

(75) Inventors: Darell Bigner, Durham, NC (US); Chien-Tsun Kuan, Cary, NC (US); Ira H. Pastan, Potomac, MD (US); Ralph Reisfeld, La Jolla, CA (US)

Correspondence Address: BANNER & WITCOFF, LTD., 1100 13th STREET, N.W., SUITE 1200, WASHINGTON, DC, 20005-4051, US

(73) Assignees: DUKE UNIVERSITY, Durham, NC (US); The United States Government as represented by the Secretary, Department of Health and Human Service, Rockville, MD (US); Scripps Clinic and Research Foundation, La Jolla, CA (US)

(21) Appl. No.: 12/418,956

(22) Filed: Apr. 6, 2009

Related U.S. Application Data
(60) Provisional application No. 61/071,110, filed on Apr. 11, 2008.

(51) Int. Cl. A61K 51/00; A61K 39/395; G01N 33/574; C07K 16/00

(52) U.S. Cl. 424/1.49; 424/130.1; 424/178.1; 435/7.23; 530/387.1; 530/391.3; 530/391.7

(57) ABSTRACT

Chondroition sulfate proteoglycans represent excellent targets for anti-tumor immunotherapy. Antibodies which target such proteoglycans can be used alone, in combinations, armed with a cytotoxic payload or unarmed. Combinations of such antibodies can target different epitopes of the proteoglycans. Internalization of the antibodies can increase the toxicity of the payloads. Single chain variable regions are especially advantageous for such anti-tumor immunotherapy.

This application claims the benefit of provisional application U.S. 61/071,110 filed Apr. 11, 2008, the contents of which are expressly incorporated herein.

### TECHNICAL FIELD OF THE INVENTION

This invention is related to the area of antibodies, antibody derivatives, and antibody combinations. In particular, it relates to antibody compositions that are useful for treating tumors.

### BACKGROUND OF THE INVENTION

Chondroitin sulfate proteoglycans are a family of glycosylated proteins. They fall into four major groups: lectcans, phosphacan/receptor type protein—tyrosine phosphatase β, leucine-rich proteoglycans, and others including neuroglycan-C and NG2. Chondroitin sulfate proteoglycans are sulfated glycosaminoglycans. They are unbranched polysaccharides comprising disaccharide unit repeats of sulfated D-glucuronic acid and N-acetyl D-galactosamine. When the polysaccharide chains are attached to proteins, they are referred to as proteoglycans. The attachments are typically via hydroxyl groups of serine residues.

Novel antibody therapeutics are emerging as the next generation of targeted drugs for cancer treatment. Immunotherapy has been explored for over a century but it is only in the past decade that the FDA has approved antibody-based, clinical drug treatments for cancer. However, the search for appropriate tumor-associated antigens is a key challenge for immunotherapeutic approaches.

Chondroitin sulfate proteoglycan (CSPG) is a glycoprotein that has been defined as a cell surface antigen for both human melanoma and glioma tumor cells, CSPG has been characterized as a molecular target for the diagnosis and treatment of melanoma and glioma using the MEL-14 IgG2a antibody. In the past, MEL-14 and MEL-14 F(ab′)₂ fragments have been successfully used for radiotherapy of human glioma xenografts and for treatment of patients with malignant glioma, melanoma and neoplastic meningitis. Additional monoclonal antibodies to chondroitin sulfate proteoglycans include 9.2.27, and MEL-5.

There is a continuing need in the art for therapeutic agents and methods for treating brain tumors and melanomas.

### SUMMARY OF THE INVENTION

According to one embodiment of the invention a single chain variable region antibody is provided which antibody specifically binds to chondroitin sulfate proteoglycans present in melanomas and gliomas and medulloblastomas.

According to another embodiment of the invention a method of treating a tumor in a human is provided. A single chain variable region antibody is administered to the human, whereby cells of the tumor are killed. The antibody specifically binds to chondroitin sulfate proteoglycans present in melanomas and gliomas and medulloblastomas.

According to still another embodiment of the invention a method is provided for determining a therapeutic plan to treat a tumor in a human. Tissue of the tumor is contacted with a single chain variable region antibody that specifically binds to chondroitin sulfate proteoglycans present in melanomas and gliomas and medulloblastomas. The amount of cells in the tissue which bind to the antibody is determined. Greater amounts of cells which bind are a positive factor to recommend using the antibody therapeutically for the patient.

According to yet another embodiment of the invention a composition comprising at least two distinct single chain variable region antibodies are provided. Each of the antibodies specifically binds to chondroitin sulfate proteoglycans present in melanomas and gliomas and medulloblastomas. They may each bind to distinct epitopes.

According to one embodiment of the invention a method of treating a tumor in a human is provided. At least two distinct single chain variable region antibodies are administered to the human, whereby cells of the tumor are killed. Each of the antibodies specifically binds to chondroitin sulfate proteoglycans present in melanomas and gliomas and medulloblastomas.

These and other embodiments which will be apparent to those of skill in the art upon reading the specification provide the art with therapeutic agents and methods for treating brain tumors and melanomas.

### BRIEF DESCRIPTION OF THE DRAWINGS

FIG. 1A-1B: SDS PAGE (FIG. 1A) and Western Blot (FIG. 1B). Analysis of the purified Immunotoxins: The purified scFv immunotoxins were analyzed by SDS PAGE Chromatography and transferred to nitrocellulose membrane for Western Blot analysis with αPE38 antibody. Samples are as follows; BSA standard 1 μg (Lane 1), Blank (Lane 2), MEL-14 PE38-KDEL (Lane 3), dsFvMel14 PE38-KDEL F8+F9 (Lane 4), dsFvMel14 PE38-KDEL F10 (Lane 5), 9.2.27 PE38-KDEL F10 (Lane 6), 9.2.27 PE38-KDEL F11 (Lane 7), dsFv9.2.27 PE38-KDEL (Lane 8), dsFvαTac PE38-KDEL (Lane 9) and BioRad Kaleidoscope Ladder (L). The ‘F’ number corresponds with the protein containing ‘fraction’ recovered following size exclusion chromatography.

FIG. 2A-2B: Flow Cytometry Analysis of the αCSPG immuntoxins reacting with D245 glioma cells. D245 cells were harvested and blocked in sterile PBS buffer containing 10% normal goat, mouse and rabbit serum to a final density of 5×10⁶ cells/mL. Approximately 5×10⁵ cells were incubated with 10 μg/mL of each of the αCSPG immuntoxins (including the negative control immunotoxin dsFv-αTac-KDEL), for 1 h at 4° C. with rotation and in the presence of 7-AAD (final concentration 0.5 μg/mL). The cells were washed with cold PBS and re-suspended in PBS-1% BSA buffer containing anti-PE38 (Rabbit polyclonal, 1:500). Following incubation for 1 h at 4° C. with rotation, the cells were washed and then incubated with anti-rabbit IgG-FITC (1:500). Finally the cells were washed and re-suspended in 0.1% PFA for acquisition on a FACSCalibur. Data is represented as MFI of viable (7-AAD -ve) cells.

FIG. 3A-3B: Cytotoxicity Analysis of the αCSPG immuntoxins reacting with D245 glioma cells. D245 cells were harvested and seeded into 96 well plates at a density of 2×104 cells/well and incubated overnight at 37° C. with 5% CO2. The cells were then treated with immunotoxin, at a concentration range from 1 μg/mL to 1 pg/mL (final concentration), for 24 h at 37° C. with 5% CO2. 1 μCi of 3H-Leucine was added to each well and further incubated for 4 h at 37° C. with 5% CO2. Following treatment at −80° C. overnight, the cells were harvested and the radioactivity counts were determined using I450 Microbeta Liquid Scintillation and Luminescence counter (Wallac, Trilux). The data was analyzed and plotted using GraphPad Prism®.

FIG. 4A-4B: Flow Cytometry Competition Analysis to determine whether Mel14 (IgG2a) and 9.2.27 (IgG1) react with the same epitope of chondroitin sulfate proteoglycan. D245 glioma (FIG. 4A) and H350 melanoma (FIG. 4B) cells were harvested and blocked in sterile PBS buffer containing 10% normal mouse serum to a final density of 5×106 cells/mL. Approximately 5×105 cells were incubated with primary antibody comprising either 4 μg of Mel14, 20 μg of 9.2.27 or 20 μg of a non reactive IgG1 antibody control. Following incubation for 30 minutes at 4° C., with rotation and in the presence of 7-AAD (final concentration 0.5 μg/mL), 2 μg of Mel14-FITC was directly added to each sample and further incubated as described. In addition, control samples included cells incubated with 2 μg of IgG2a-FITC (negative) or 2 μg of Mel14-FITC. Following incubation, the cells were washed with cold PBS and re-suspended in 0.1% PFA for acquisition on a FACSCalibur. Data is represented as MFI of viable (7-AAD -ve) cells.

FIG. 5A-5B. Flow cytometry analysis of melanoma line Malme3M p40 using Mel14-FITC (FIG. 5A) and αCSPG scFv (FIG. 5B).

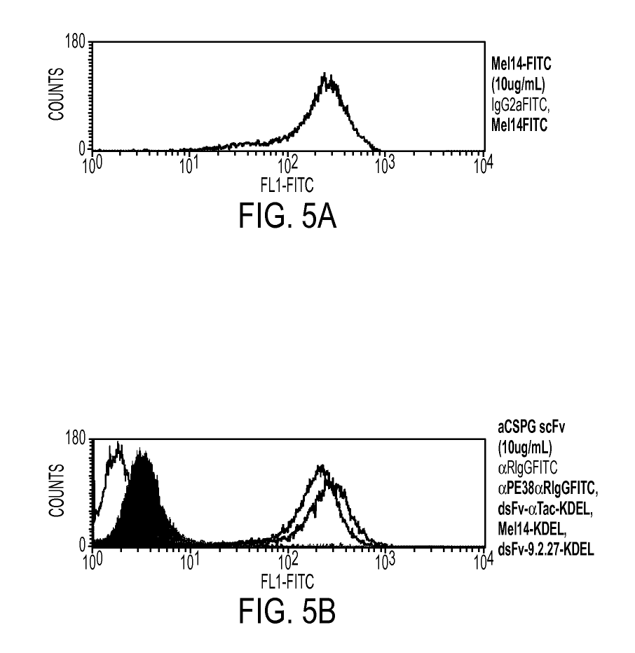

A sequence listing forms part of this disclosure.

### DETAILED DESCRIPTION OF THE INVENTION

Monoclonal antibodies Mel-14, 9.2.27, and MEL-5 have some excellent properties for treating human glioma, melanoma, medulloblastoma, and neoplastic meningitis. All three antibodies are reactive with tumor-associated chondroitin sulfate proteoglycan. However, all of their properties are not optimal for use in human tumor therapy. All three are mouse monoclonal antibodies produced by hybridomas. These antibodies can be “converted” into other forms, for example, humanized, chimeric, and single chain variable region antibodies. These conversions are well known in the art and typically involve cloning of the antibody encoding genes from the hybridomas which produced the mouse monoclonal antibodies. When the V_{H} or V_{L} or the CDR region sequences remain the same as in the original monoclonal antibody, then the converted form likely retains binding specificity of the original monoclonal antibody. Surprisingly, such converted forms can be internalized by cancer cells.

Such antibodies and “converted” antibodies can be bound, either covalently or non-covalently, to other useful moieties. For example, they can be conjugated to radionuclides or radioactive moieties. They can be joined to a biological toxin, such as Pseudomanas exotoxin A, ricin, or diphtheria toxin. They can be conjugated to chemotherapeutic agents. They can be joined to other antibodies. Attachments of the antibodies to other moieties can occur by means of genetic engineering, if the other moiety is a protein, so that a fusion protein is produced in a host cell. The attachments may be done chemically, in vitro. The attachments may be covalent or non-covalent. Non-covalent attachments preferably use strong, biological, specific-binding pairs to achieve strong attachments, such as avidin/biotin. For diagnostic purposes, the antibodies can be attached to chromophores, or other easily detectable moieties.

Other moieties which can be attached to the antibodies include those which provide additional beneficial properties. For example, a KDEL (lys-asp-glu-leu) tetra-peptide can be added at the carboxy-terminus of the protein to provide retention in the endoplasmic reticulum. Variants such as DKEL, RDEL, and KNEL which function similarly can also be used.

Tumors which can be treated include human glioma, melanoma, medulloblastoma, and neoplastic meningitis, or other tumors. It may be desirable to determine the expression of the antigen in the tumor prior to therapy. When the antigen is expressed by the tumor, that is a positive factor for treating with the converted forms of the antibodies for the antigen.

Combinations of antibodies and converted forms of the antibodies can be used for enhanced therapeutic effect. Preferably the combinations bind to different epitopes and/or are derived from different monoclonal antibodies. In some embodiments the combinations of antibodies contain different effector molecules, such as different toxins or chemotherapeutic agents or radiotherapeutic agents.

Antibodies and antibody constructs and derivatives can be administered by any technique known in the art. Compartmental delivery may be desirable to avoid cytotoxicity for normal tissues. Suitable compartmental delivery methods include, but are not limited to delivery to the brain, delivery to a surgically created tumor resection cavity, delivery to a natural tumor cyst, and delivery to tumor parenchyma.

MEL-14 antibodies were first described in Carrel et al., “Common human melanoma-associated antigen(s) detected by monoclonal antibodies,” Cancer Research 40: 2523-2528, 1980, and later used to make F(ab′)₂. See Cope et al., “Enhanced delivery of a monoclonal antibody f(ab′)₂ fragment to subcutaneous human glioma xenografts using local hyperthermia,” Cancer Research 50, 1803-1809, 1990. These are available commercially from AbCam. MEL-5 antibodies are available commercially from Covance Research Products, Inc. Antibody 9.2.27 was first described in 1981. See Morgan et al., “Production and characterization of monoclonal antibody to a melanoma specific glycoprotein,” Hybridoma 1: 27-36, 1981. It is available commercially from from the Santa Cruz Biotechnology, Inc., Santa Cruz, Calif.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### MEL-14 scFv Immunotoxin

Recent flow cytometry analysis of neurospheres, isolated from brain tumor stem cells of glioma xenografts, has renewed our interest in CSPG as a tumor marker and molecular target for immunotherapy. Results show that in many of the samples analyzed, a significant percentage (>70%) of the viable population demonstrated expression of CSPG; with the overall range accommodating between 30 and 100%. In order to develop the MEL-14 single chain variable fragment (scFv) as a potential immunotherapeutic we have expressed the scFv as a fusion with the Pseudomonas exotoxin, PE38, containing the four carboxyl amino acid sequence, KDEL. The immunotoxin has been recovered from bacterial inclusion bodies and purified using ionic exchange chromatography. Flow cytometry analysis has confirmed that purified MEL-14 scFv immunotoxin binds D54MG glioma cells.

### Example 2

### Generation of Mel-14-PE35KDEL and 9.2.27-PE35KDEL

Expression and Purification of PE35KDEL. To express PE35KDEL protein, a plasmid pRK35K was used to transform Escherichia coli BL21 (λDE3) for expression. Periplasm was prepared and PE35KDEL was purified by sequential Q-Sepharose ion exchange chromatography, Mono Q ion exchange chromatography, and TSK G3000SW size exclusion chromatography (TosoHaas, Montgomeryville, Pa.).

Antibody Conjugation. Antibody (2 mg) in 500 μl PBS/1 mM EDTA was reacted for 1 h at 37° C. with a 7-10-fold molar excess of 2-iminothiolane (Pierce Chemical Company, Rockville, Ill.) and desalted on a PD10 column (Pharmacia Biotech AB). Dithionitrobenzoate (50 μl of a 10 mM solution; Pierce Chemical Company) was then added. Absorbance measurements at 412 nm showed that 1-2 mol of free thiol/mol antibody were produced by the reaction with 2-iminothiolane. The antibody was desalted again on a PD10 column. Two mg PE35KDEL were treated for 30 min with 20 mM DTT, desalted on a PD10 column, and mixed with the antibody. The mixture was incubated overnight at 10° C. and 1:1 antibody:PE35KDEL conjugates were purified by TSK size exclusion chromatography and Mono Q ion exchange chromatography. The concentration of the immunotoxins was determined by absorbance measurements at 280 nm, using ε=1.43 mg/mg·cm, or with Coomassie Plus Protein Assay Reagent (Pierce Chemical Company) using BSA as standard.

Cytotoxicity Assay. Cytotoxicity on cultured cell lines was assayed by inhibition of protein synthesis as described previously. Cells were plated in 96-well plates at 2×10⁴ cells in 180 μl of complete medium per well 24 h before the assay. Immunotoxins were serially diluted (0.01-1000 ng/ml) in PBS containing 0.2% bovine serum albumin (BSA; 0.2% BSA/PBS), and 20 μl of diluted toxin was added to each well. Plates were incubated for 20 h at 37° C. and then pulsed with 1 μCi/well of L-[4,5-³H]-leucine (Amersham Biosciences, Little Chalfont, Buckinghamshire, UK) in 20 μl of 0.2% BSA/PBS for 2 h at 37° C. Radiolabeled cells were captured on filter-mats and counted in a MicroBeta scintillation counter (PerkinElmer, Shelton, Conn.). The cytotoxic activity of immunotoxin was defined by IC₅₀, which was the toxin concentration that suppressed incorporation of radioactivity by 50% as compared to the cells that were not treated with toxin.

### Example 3

### Human Clinical Studies with Mel-14 F(ab′)₂

Mel-14 is a murine anti-melanoma IgG2a monoclonal antibody that was developed by Carrel et al (1980). It recognizes a high molecular weight chondroitin sulfate proteoglycan antigen of approximately 240 kDa (gp 240) associated with human gliomas, melanomas, and other tumors. It reacts with most melanoma cell lines as well as with a high percentage of glioma, neuroblastoma, and medulloblastoma lines. In tissue immunohistochemistry it has been shown to bind to tumor and endothelial cells in biopsy samples of human GBM and to most primary and metastatic melanomas. It does not react with normal human brain (Behnke et al, 1987; Behnke et al, 1988; Schreyer et al, 1986). It localizes specifically in paired-label studies to human melanoma xenografts in athymic mice (Buchegger et al, 1986). The F(ab′)₂ fragment of Mel-14 also localizes specifically in paired-label studies to human glioma xenografts in athymic mice and has been administered and shown to localize specifically and similarly in human gliomas in the brain of patients (Behnke et al, 1987; Behnke et al, 1988; Zalutsky et al, 1990). Doses of up to 20 mg of the Mel-14 F(ab′)₂ fragment, trace labeled with ¹³¹I, ¹²¹I, or ¹²³I, have been administered intravenously or in the carotid artery to glioma patients with no toxicity. Therapeutic efficacy of systemically administered ¹³¹I-labeled Mel-14 F(ab′)₂ has been shown by survival prolongation in mice bearing intracerebral human D54 MG xenografts following administration of up to 2 mCi per animal of radiolabeled Mel-14 F(ab′)₂ (Colapinto et al, 1990).

Phase I/II Intrathecal ¹³¹I-Mel-14 F(ab′)₂ for Melanoma and Other Neoplastic Meningitis (NM)—We have administered 21 treatments of ¹³¹I-labeled Mel-14 F(ab′)₂ to 20 patients with NM. Ten patients had melanoma NM and 4 had GBM.

### Example 4

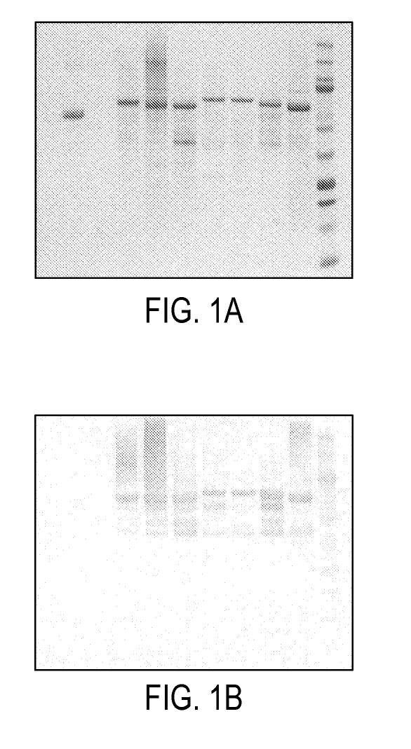

We cloned and assembled VH and VL of the 9.2.2.7 scFv into the PE38-KDEL expression vector. We mutagenized the DNA clones to create dsFv-αTac-KDEL (negative control scFv), dsFv-MEL-14-KDEL and dsFv-9.2.27-KDEL immunotoxins. We purified the five different immunotoxins; MEL-14 PE38-KDEL, dsFvMEL-14 PE38-KDEL, 9.2.27 PE38-KDEL, dsFv9.2.27 PE38-KDEL and dsFvαTac PE38-KDEL (negative control antibody) as shown in FIG. 1A-1B.

### Example 5

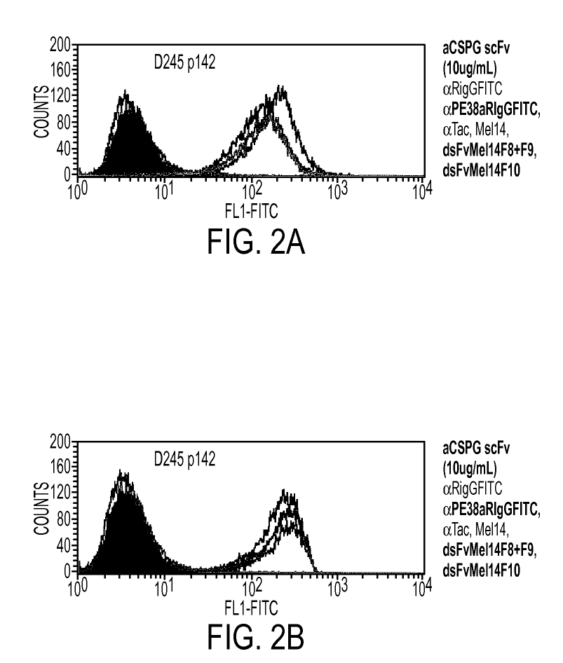

Flow cytometry techniques were used to demonstrate positive reactivity of all purified αCSPG immunotoxins with cultured melanoma (H350, A375, Malme3M, SKMel2 and SKMel5) and glioma cell lines (D54, D79, D245, D247, D263 and TB2620/F3). FIG. 2 shows reactivity with D245 glioma cells.

### Example 6

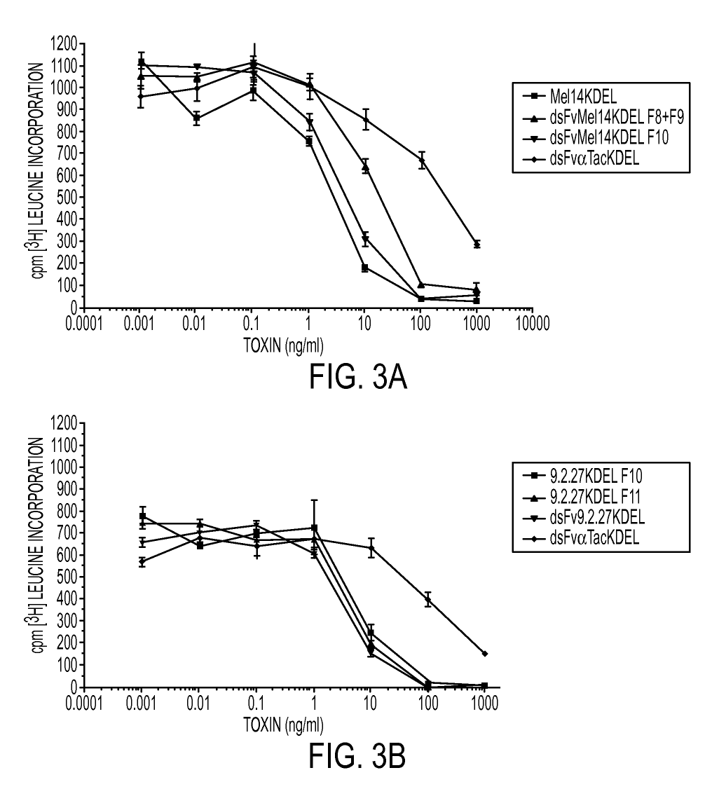

Cytotoxicity experiments were performed using a panel of melanoma (H350, A375, Malme3M, SKMel2 and SKMel5) and glioma (D54, D263, TB2620, D245, D79, D247) cell lines. FIG. 3 shows the cytotoxicity results produced with D245 glioma cells and Table 1 summarizes the IC₅₀ values for all cell lines tested.

TABLE 1
Summary of the IC₅₀ (ng/mL) cytotoxicity values for the glioma and melanoma cell lines tested.

| Cell | Tumor | Mel14- | dsFv | dsFv | | | dsFv |
|---|---|---|---|---|---|---|---|
| Line | Type | KDEL | Mel14F8F9 | Mel14F10 | 9.2.27F10 | 9.2.27F11 | 9.2.27 |
| D245 | Glioma | 4 | 28 | 6 | 8 | 6 | 5 |
| D54 | Glioma | 50 | 60 | 270 | 55 | 70 | 100 |
| D247 | Glioma | 100 | 190 | 600 | Not Killing | 80 | 60 |
| D79 | Glioma | 200 | 300 | 700 | 220 | 100 | 55 |
| TB2620 | Glioma | 150 | 2000 | 220 | 500 | 180 | 130 |
| D263 | Glioma | Not Killing | Not Killing | Not Killing | Not Killing | Not Killing | Not Killing |
| Malme3M | Melanoma | 1 | 5 | 1.8 | 18 | 20 | 8.5 |
| H350 | Melanoma | 8 | 40 | 13 | 20 | 20 | 12 |
| SKMel2 | Melanoma | 36 | 150 | 42 | 2400 | 700 | 200 |
| SKMel5 | Melanoma | 60 | 180 | 50 | 350 | 200 | 60 |
| A375 (Expt 1) | Melanoma | Not Killing | Not Killing | Not Killing | Not Killing | Not Killing | Not Killing |
| A375 (Expt 2) | Melanoma | 500 | 1600 | 800 | 4500 | 4500 | 200 |
| A375 (Expt 3) | Melanoma | 450 | 800 | 350 | Not Killing | 600 | 200 |

### Example 7

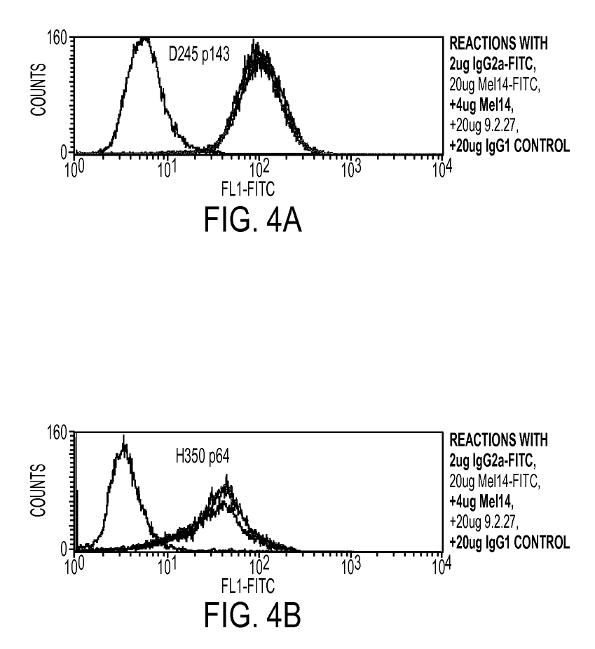

Flow cytometry analysis was performed using D245 glioma and H350 melanoma cells. Results show that the antibodies did not compete with each other, demonstrating that they react with a different epitope of the chondroitin sulfate proteoglycan surface molecule (FIG. 4).

### Example 8

Both MEL-14 PE38-KDEL and 9.2.27 PE38-KDEL immunotoxins have shown very promising in vitro therapeutic biological activity against both glioma and melanoma cell lines. The cytotoxic IC₅₀ (ng/mL) values are within the acceptable range of efficacy, less than 10 ng/mL, for cell lines D245, H350 and Malme3M (refer to Table 1). Disulphide stabilization does not have a significant affect on the biological stability or activity of the Mel14 scFv antibody. However, the dsFv 9.2.27 scFv is more potent than the non-disulphide stabilized protein. It is particularly important to note that because the two antibodies react with different epitopes of the chondroitin sulfate proteoglycan molecule (refer to FIG. 4), both Mel14 and 9.2.27 scFv immunotoxins they represent distinct and combined potential therapeutics for glioma and melanoma.

We claim:

1. A single chain variable region antibody which specifically binds to chondroitin sulfate proteoglycans present in melanomas and gliomas and medulloblastomas.

2. The single chain variable region antibody of claim 1 which is cloned from a hybridoma producing a monoclonal antibody MEL-14.

3. The single chain variable region antibody of claim 1 which is cloned from a hybridoma producing a monoclonal antibody MEL-5

4. The single chain variable region antibody of claim 1 which is cloned from a hybridoma producing a monoclonal antibody 9.2.27

5. The single chain variable region antibody of claim 1 which is covalently linked to a cytotoxic agent selected from the group consisting of a toxin, a chemotherapeutic agent, and a radionuclide.

6. The single chain variable region antibody of claim 5 wherein the agent is a toxin which is produced as a fusion protein with the single chain variable region antibody.

7. The single chain variable region antibody of claim 5 wherein the agent is a form of Pseudomanas exotoxin A.

8. The single chain variable region antibody of claim 5 further comprising a KDEL peptide.

9. The single chain variable region antibody of claim 1 which has a VH sequence from a hybridoma producing MEL-14 antibodies.

10. The single chain variable region antibody of claim 1 which as a VL sequence from a hybridoma producing MEL-14 antibodies.

11. The single chain variable region antibody of claim 1 which has CDR1, CDR2, and CDR3 regions from a hybridoma producing MEL-14 antibodies.

12. The single chain variable region antibody of claim 1 which has a VH sequence from a hybridoma producing MEL-5 antibodies.

13. The single chain variable region antibody of claim 1 which as a VL sequence from a hybridoma producing MEL-5 antibodies.

14. The single chain variable region antibody of claim 1 which has CDR1, CDR2, and CDR3 regions from a hybridoma producing MEL-5 antibodies.

15. The single chain variable region antibody of claim 1 which has a VH sequence from a hybridoma producing 9.2.27 antibodies.

16. The single chain variable region antibody of claim 1 which as a VL sequence from a hybridoma producing 9.2.27 antibodies.

17. The single chain variable region antibody of claim 1 which has CDR1, CDR2, and CDR3 regions from a hybridoma producing 9.2.27 antibodies.

18. A method of treating a tumor in a human, comprising:
administering a single chain variable region antibody according to claim 1 or 5 to the human, whereby tumor cells are killed.

19. The method of claim 18 wherein the tumor is a glioma.

20. The method of claim 18 wherein the tumor is a brain tumor.

21. The method of claim 18 wherein the tumor is a medulloblastoma.

22. The method of claim 18 wherein the tumor is a melanoma.

23. The method of claim 18 wherein the administering is directly to the central nervous system.

24. The method of claim 18 wherein the administering is directly to the brain.

25. The method of claim 18 wherein the administering is directly to a surgically-created tumor resection cavity.

26. The method of claim 18 wherein the administering is directly to a natural tumor cyst.

27. The method of claim 18 wherein the administering is directly to tumor parenchyma.

28. A method of determining a therapeutic plan to treat a tumor in a human, comprising:
contacting tissue of the tumor with an antibody according to claim 1;
determining amount of cells in the tissue which bind to the antibody, wherein greater amounts of cells which bind are a positive factor to recommend using the antibody therapeutically for the patient.

29. A composition comprising at least two distinct single chain variable region antibodies according to claim 1.

30. A composition comprising at least two distinct single chain variable region antibodies according to claim 5.

31. A composition comprising at least two distinct single chain variable region antibodies according to claim 7.

32. A composition comprising at least two distinct single chain variable region antibodies according to claim 8.

33. A method of treating a tumor in a human, comprising:
administering at least two distinct single chain variable region antibodies according to claim 1, 5, 7, or 8 to the human, whereby tumor cells are killed.
